(19) United States
(12) Patent Application Publication
(10) Pub. No.: US 2018/0193287 A1
(43) Pub. Date: Jul. 12, 2018

(54) HOPS-BASED SUBSTANCE AND USE OF THE SUBSTANCE

(71) Applicant: John I. Haas, Inc., Washington, DC (US)

(72) Inventor: Claus Hellerbrand, Regensburg (DE)

(21) Appl. No.: 15/740,281

(22) PCT Filed: Jul. 2, 2015
(86) PCT No.: PCT/EP2015/065107

§ 371 Date: Dec. 27, 2017

(51) Int. Cl. A61K 31/121; A61K 9/00; A61P 1/16; A61P 3/04; A23L 33/105

(52) U.S. Cl. CPC A61K 31/121; A61K 9/0053; A61P 1/16; A61P 3/04; A23V 2250/2132; A23V 2002/00; A23V 2200/328; A23V 2200/332; A23L 33/105

(57) ABSTRACT

The invention relates to a hops-based substance which comprises a mixture of (a) xanthohumol with the formula (1) and (b) at least one isomerized hops acid, preferably iso-alpha acid, in each case in a concentrated or isolated form. It has surprisingly been shown that a substance in which xanthohumol and iso-alpha acid are present in combination and in which each substance is present in a concentrated or isolated form in the combination substantially increases the effectiveness in conjunction with the prevention and/or control of liver diseases or liver damage when compared to xanthohumol alone. The inflammation- and fibrosis-inhibiting effect of xanthohumol and the inhibition of the fatty changes of liver cells is thus surprisingly increased by the presence of iso-alpha acid.

The present invention relates to a hop-based substance which comprises, as active substances, concentrated or isolated xanthohumol and an isomerized hop acid, preferably concentrated or isolated iso-alpha-acid, in combination. Furthermore, the present invention relates to the use of the abovementioned substance for preventing and/or controlling liver disease or liver damage, and also obesity or diabetes.

### TECHNOLOGICAL BACKGROUND

Besides alcohol consumption, obesity and diabetes are currently responsible for the majority of liver disease. The latter is also referred to as nonalcoholic fatty liver disease (NAFLD). Their incidence is still on the rise, mainly due to eating habits. In total, liver disease, or liver damage, has become an important economical problem.

Attempts are therefore being made to find potential therapies, to prevent liver disease or liver damage in the first place or, if they already exist, to alleviate them.

Xanthohumol is a prenylated plant polyphenol which is assigned to the chalcones and has to date exclusively been detected in hops. Xanthohumol has been known for some time for its health-promoting activities, especially in connection with liver disease.

Iso-alpha-acids cannot be found in natural hops. Extraction allows soft and hard resins to be obtained from the hop plant. Soft resins are divided into humulones, i.e. alpha-acids, and the structurally related lupulones, i.e. beta-acids. Alpha-acids are highly unstable. During wort boiling, which is part of beer production, isomerization of the alpha-acids gives rise to the highly bitter iso-alpha-acids, i.e. the isohumulones. They are used for adjusting the beer's bitterness during the brewing process.

### PRIOR ART DOCUMENTS

WO 2008/077 618 A1 discloses the use of xanthohumol or isoxanthohumol as active substance for preventing and/or controlling liver disease. It has been found that liver disease or liver damage due to obesity (overweight) or diabetes can effectively be prevented by the regular intake of xanthohumol over a prolonged period. Moreover, it has been demonstrated that xanthohumol has a positive effect on other forms of liver damage, too.

### PROBLEM OF THE PRESENT INVENTION

It is a problem of the present invention to provide a novel substance which ensures an activity with regard to the prevention and/or control of liver disease or liver damage and/or of obesity and/or diabetes which is improved in comparison with the prior art. Another problem of the present invention is to provide novel improved uses of the abovementioned substance.

### SOLUTION ACCORDING TO THE INVENTION

The problem is solved by a substance as per the features of claim 1. Expedient embodiments of the present invention are claimed in the subsequent claims.

The substance according to the invention comprises a base substance, in which xanthohumol and iso-alpha-acid are present in combination. Here, each part-substance is preferably present in each case in concentrated or isolated form, and preferably as a mixture of the two part-substances. Surprisingly, it has been shown that the activity of xanthohumol in connection with the prevention and/or control of liver disease or liver damage or of obesity or of diabetes can be increased substantially by means of a substance in which xanthohumol and iso-alpha-acid in combination, with each abovementioned substance being present in each case in concentrated or isolated form. The antiinflammatory and anti-obesity action of xanthohumol is surprisingly and unexpectedly increased by the presence of iso-alpha-acid. The combination makes it possible to reduce the xanthohumol concentration. This is advantageous since, when concentrating xanthohumol, the so-called 8-prenylnaringenin, which is likewise present in hops, is usually also concentrated. This is a substance with an undesired estrogen activity. The invention therefore has the advantage that it is possible to reduce the amount of xanthohumol in the prophylaxis and/or treatment.

Expediently, the amount of iso-alpha-acid in the mixture exceeds the xanthohumol. The amount is preferably the content in % by weight, based on the respective substance or, in mathematical terms, based on the respective pure substance.

The substance according to the invention especially advantageously comprises xanthohumol and iso-alpha-acid in a weight ratio of from 0.5 to 10, preferably from 0.8 to 8, especially preferably from 1 to 5, % by weight. These weight ratio data refer to the actual weight ratio in % by weight or (in mathematical terms) to the amount of the respective pure substance.

Preferably, the substance according to the invention is designed such that it is suited to oral administration.

In particular, the substance according to the invention may comprise a pharmaceutically acceptable carrier, which makes possible the application of the substance to or in the human body.

The present invention furthermore relates to the use of a substance as per patent claims 1-7 for the preparation of a product for the prevention and/or control of liver disease or liver damage.

It has emerged that the combination according to the invention of the two substances has an especially good activity in particular in the case of cirrhosis or fibrosis of the liver, both in a prophylaxis and as medicament for treatment.

It has furthermore emerged that the combination according to the invention of the two substances has a particularly good activity especially also in the case of inflammation of the liver tissue and in fatty degeneration of the liver, both in a prophylaxis and as a medicament for acute treatment.

In addition, it has emerged that the combination according to the invention of the two substances has a particularly good activity even in the case of obesity and/or diabetes.

Also, the combination according to the invention provides an increased activity in the prophylaxis of nonalcoholic steatosis hepatitis (NASH), both in the case of prophylaxis and also as medicament for acute treatment.

Likewise, the substance according to the invention provides an increased activity in the prophylaxis of alcohol-induced liver damage, both in prophylaxis and as medicament for acute treatment.

Owing to the acceptability of its components, the substance may especially preferably be ingested prophylactically over a prolonged period so as to efficiently counteract liver disease or liver damage of the aforementioned types in a preventative fashion.

Owing to the combination, it is possible to reduce the concentration of the individual component xanthohumol, and thus to diminish the potential side effects of xanthohumol as individual substance.

The claimed use has the advantage that, using a natural active substance, liver disease can efficiently be avoided or controlled both preventively and by means of treatment.

No side effects are known for iso-alpha-acids. Therefore, no side effects should be expected either for a combination of xanthohumol and iso-alpha-acids.

Furthermore, it should be expected that the risk of any side effects which are not known to date can be reduced further if the substances can efficiently be employed in combination, in a lower concentration. This allows xanthohumol to be employed especially efficiently over a prolonged period for preventing and/or treating chronic liver disease, in particular chronic liver disease.

The combination of xanthohumol and iso-alpha-acids is also well suited to the prevention or acute treatment of cirrhosis or fibrosis of the liver. In studies it has, surprisingly, emerged that xanthohumol inhibits metabolic mechanisms which are of very particular importance for liver damage caused by obesity (overweight) and diabetes. Obesity and diabetes are responsible for the majority of cirrhoses of the liver. The trend is increasing. Chronic liver disease in total has by now become an important economical problem. An effective prophylactic protection for the entire population, without side effects, can be established by the continuous intake of a combination of xanthohumol and iso-alpha-acids.

Furthermore, studies have shown that a combination of xanthohumol and iso-alpha-acids has antiviral properties and has very good activity against hepatitis, in particular against hepatitis B and hepatitis C. Hepatitis B or hepatitis C is the most frequent cause for the development of chronic liver disease. Epidemiological studies in Germany have shown that approximately 2% of the population suffer from chronic hepatitis B or hepatitis C. This is therefore also a problem of central sociological importance. The prophylactic intake of xanthohumol therefore allows firstly effective reduction of the amount of hepatitis diseases, i.e. hepatitis B and/or C diseases, and, secondly, favorable influencing of the course of a preexisting hepatitis disease.

Currently no guaranteed forms of therapy are available for the treatment of fibrosis of the liver. An inhibition or a stop of the progression of fibrosis can only be achieved by eliminating the damaging cause, that is to say for example in the case of hepatitis virus infection by eliminating the hepatitis viruses. However, eliminating the damaging cause only succeeds in some of the patients with chronic liver disease, or is currently generally not possible in patients with genetic liver disease. In the case of hepatitis virus infections, the use of medicaments with potent side effects has been required to date. Even if such medicaments are being used, virus is eliminated successfully only in some of the patents. It is welcome that the use of xanthohumol may be able to remedy this.

Finally, a combination of xanthohumol and iso-alpha-acids also has anticarcinogenic activity. For cancer of the liver, or hepatocellular carcinoma (HCC), there is currently no guaranteed therapy available besides the surgical method which would improve the patients' survival. Currently, surgical removal results in success only in a minor number of HCC patients, since the HCC will in most cases, when a diagnosis is being made, already be unduly large or have developed metastases. Owing to the increased activity, the combination of xanthohumol and iso-alpha-acids can also be employed therapeutically against cancer of the liver.

In addition, a combination of xanthohumol and iso-alpha-acids can be employed prophylactically precisely in persons at high risk (genetic risk, obesity, diabetics).

As regards the administration, a use is provided in accordance with the invention such that the xanthohumol and the iso-alpha-acids are administered as active component of a pharmaceutical composition together with a pharmaceutically acceptable carrier such as, for example, mannitol, sucrose, lactose, glucose, fructose and/or maltose and the like.

Owing to the absence of side effects, a combination of xanthohumol and iso-alpha-acids is very particularly suited to being added, as active substance, to a foodstuff and/or admixed to a beverage.

As regards the obtaining of xanthohumol from hop plants, reference is made to EP 0 679 393 B 1 and EP 1 543 834 A 1 in their entirety. Alpha-acids are extracted from the hop plant and subsequently isomerized to give iso-alpha-acid. Iso-alpha-acid has the following chemical formula:

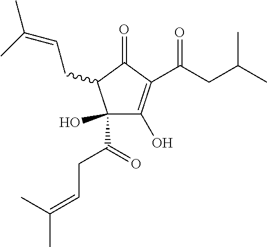 [SMILES: CC(C)=CCC(=O)[C@@]1(O)C(O)=C(C(=O)CC(C)C)C(=O)C1CC=C(C)C]

### EXAMPLE 1

An exemplary composition of a pharmaceutical is given hereinbelow.

Powder mixture for direct compression

| | |
|---|---|
| Xanthohumol (pure substance) | 2 g |
| Iso-alpha-acid | 8 g |
| Microcrystalline cellulose | 10% by weight |
| Sodium carboxymethyl starch | 3% by weight |
| Highly-disperse silica | 1% by weight |
| Magnesium stearate | 1% by weight |
| Tablettose (lactose monohydrate) | to 100% by weight |

### EXAMPLE 2

An exemplary composition of a foodstuff with xanthohumol added as active substance is given hereinbelow.
Xanthohumol (pure substance in the form of a powder) 150 mg per 200 ml
Iso-alpha-acid 350 mg per 200 ml
Dairy product (creamy, for example yoghurt)

Owing to the admixture into a creamy foodstuff, which can be carried out in a simple manner, the above composition for a foodstuff allows an administration which is optimally suited to the required amount of xanthohumol.

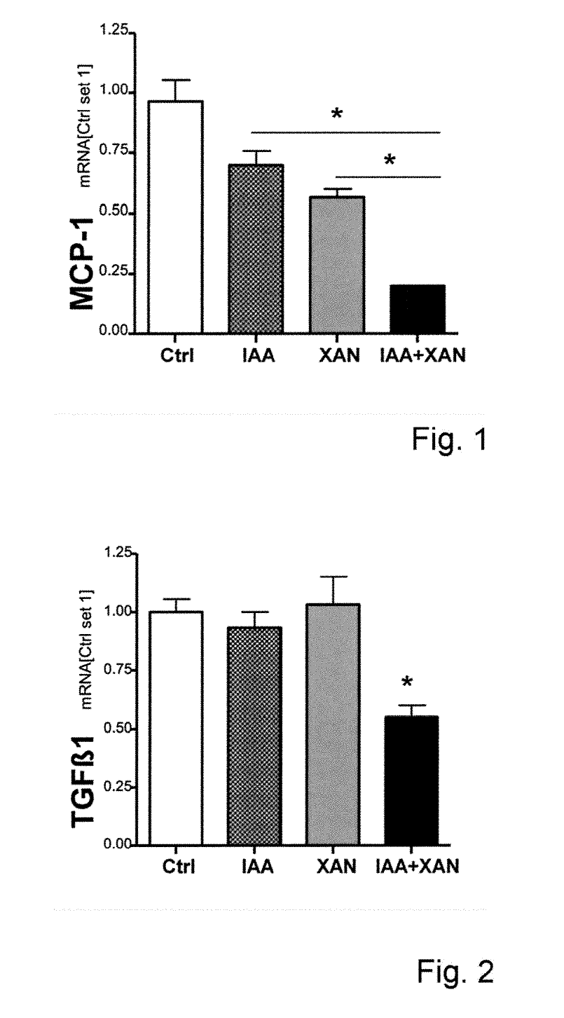

The diagram as per FIG. 1 shows the inhibition of MCP-1 by a combination of xanthohumol and iso-alpha-acid. MCP-1 is a chemokine which plays a central role in the hepatic inflammatory reaction and fibrosis, including of nonalcoholic fatty liver disease (NAFLD). Human hepatic stellate cells (HSC) were stimulated for 24 hours with iso-alpha-acid (IAA; 10 μg/ml), with xanthohumol (5 μmol) and with a combination comprising xanthohumol (5 μmol) and iso-alpha-acid (10 μg/ml) in a cell culture model. Control cells (CTRL), i.e. unstimulated cells, were likewise studied. After 24 hours, the cells' RNA was isolated, and the MCP-1 expression was determined by means of quantitative PCR analyses. The significance level is at P<0.05.

The diagram as per FIG. 1 shows that a combination of xanthohumol and iso-alpha-acid in comparison with xanthohumol alone and iso-alpha-acid alone results in a substantial reduction of MCP-1. Surprisingly, the reduction for the combination of xanthohumol and iso-alpha-acid is more than twice as pronounced in comparison with the individual substances.

The diagram as per FIG. 2 shows the inhibition of TGF-β1 by a combination of xanthohumol and iso-alpha-acid. TGF-β1 is one of the most important profibrogenic factors in all types of liver disease. In a further cell culture model, human hepatic stellate cells (HSC) were stimulated for 24 hours with iso-alpha-acid (IAA; 10 μg/ml), with xanthohumol (5 μmol) or with a combination of xanthohumol (5 μmol) and iso-alpha-acid (10 μg/ml). Control cells (CTRL), i.e. nonstimulated cells, were likewise studied. After 24 hours, the cells' RNA was isolated, and the expression of TGF-β1 was determined by means of quantitative PCR analyses. The significance level is at P<0.05.

The diagram as per FIG. 2 shows that a combination of xanthohumol and iso-alpha-acid in comparison with xanthohumol alone or iso-alpha-acid alone results in a substantial reduction of TGF-β1.

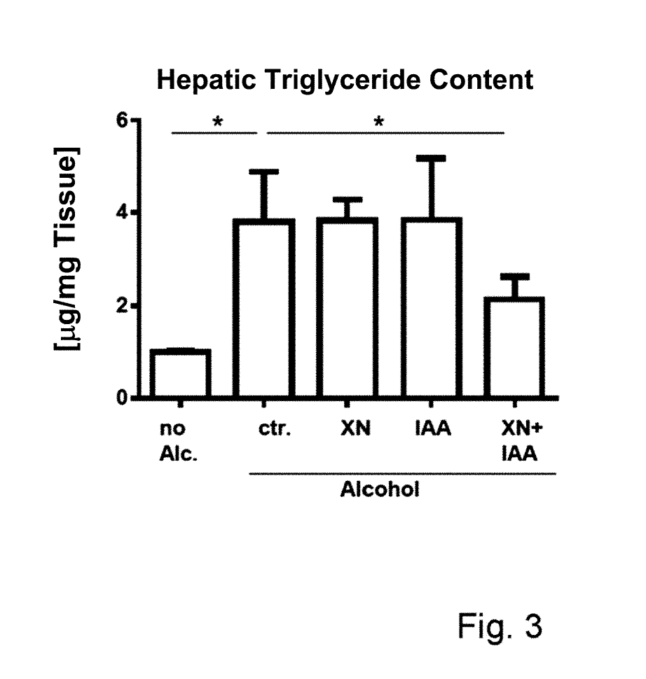

The diagram as per FIG. 3 shows data which show the synergistic effect of a combination of xanthohumol and iso-alpha-acid in an in-vivo model. A model used was acute liver damage in mice as a result of alcohol. Alcohol was applied to the mice by gavage (6 g/kg body weight). Furthermore, xanthohumol (XN; 5 mg/kg body weight), iso-alpha-acid (IAA; 25 mg/kg body weight) and a combination of xanthohumol (5 mg/kg body weight) and iso-alpha-acid (25 mg/kg body weight) were simultaneously applied in the experimental groups. After 12 hours the animals were sacrificed, the liver tissue was removed, and the triglyceride (TG) obtention therein was determined. A control group of untreated mice (no-Alc) was also tested. The results are shown in FIG. 3. In the “alcohol group”, a significant increase in TG can be seen in comparison with the “non-alcohol group”. In the xanthohumol (XN)-only groups and the iso-alpha-acid (IAA)-only groups, the TG content does not differ significantly from the “alcohol group”.

In the combined xanthohumol and iso-alpha-acid use group, in contrast, the TG content is, in contrast, significantly lower than in the “alcohol group”. Again, the significance level is P<0.05. CTR stands for a group of alcohol-treated mice without addition of xanthohumol, iso-alpha-acid or a combination of xanthohumol and iso-alpha-acid.

1. A substance, preferably a hop-based substance, comprising a combination of
(a) xanthohumol with the formula 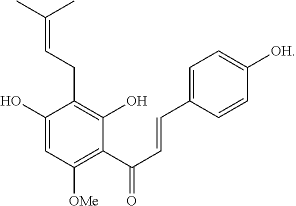 [SMILES: COc1cc(O)c(CC=C(C)C)c(O)c1C(=O)/C=C/c1ccc(O)cc1]
and
(b) an isomerized alpha-acid.

2. The substance of claim 1, wherein the isomerized alpha-acid is present in the substance in each case in concentrated or isolated form.

3. The substance of claim 1, wherein the xanthohumol is present in the substance in each case in concentrated or isolated form or in synthetic form.

4. The substance of claim 1, wherein the amount of isomerized hop acid in the mixture exceeds the amount of xanthohumol.

5. The substance of claim 1, wherein the amount of xanthohumol to isomerized hop acid lies at a ratio of 0.5 to 10, preferably 0.8 to 8, especially preferably 1 to 5% w/w.

6. The substance of claim 1, wherein the substance can be administered directly orally.

7. The substance of claim 1, wherein the substance comprises a pharmaceutically acceptable carrier.

8. The substance according to claim 1, wherein the substance is included in a preparation for the prevention and/or control of inflammatory changes of the liver tissue.

9. The substance according to claim 1, wherein the substance is included in a preparation for the prevention and/or control of cirrhosis or fibrosis of the liver.

10. The substance according to claim 1, wherein the substance is included in a preparation for the prevention and/or control of a nonalcoholic fatty liver disease (NAFLD).

11. The substance according to claim 1, wherein the substance is included in a preparation for the prevention and/or control of an alcohol-induced liver damage.

12. The substance according to claim 1, wherein the substance is included in a preparation for the prevention and/or control of obesity and/or diabetes.

13. The substance according to claim 9 in a dosage corresponding to a ratio of 0.001-100 mg/kg body weight, preferably 0.001-50 mg/kg body weight, especially preferably 0.01-10 mg/kg body weight.

14. A food supplement, comprising a substance according to claim 1.

15. The substance of claim 1 comprising isomerized alpha-acid present in concentrated or isolated form and xanthohumol present in concentrated or isolated form or in synthetic form.

16. The food supplement of claim 14 comprising isomerized alpha-acid present in concentrated or isolated form and xanthohumol present in concentrated or isolated form or in synthetic form.

17. A method of treatment comprising administering a hop-based substance comprising a combination of:
(a) xanthohumol with the formula 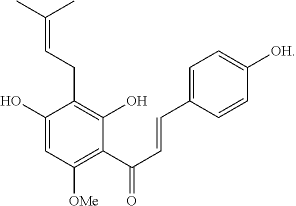 [SMILES: COc1cc(O)c(CC=C(C)C)c(O)c1C(=O)/C=C/c1ccc(O)cc1]
and
(b) an isomerized alpha-acid to a subject in need thereof.

18. The method of claim 17, wherein the subject is in need of at least one of prevention or control of inflammatory changes of the liver tissue, cirrhosis or fibrosis of the liver, nonalcoholic fatty liver disease (NAFLD), alcohol-induced liver damage, and obesity and/or diabetes.

19. The method of claim 17, wherein the substance comprises isomerized alpha-acid present in concentrated or isolated form and xanthohumol present in concentrated or isolated form or in synthetic form.

20. The method of claim 19, wherein the amount of isomerized hop acid present exceeds the amount of xanthohumol present.
